(19) United States
(12) Patent Application Publication
(10) Pub. No.: US 2011/0098549 A1
(43) Pub. Date: Apr. 28, 2011

(54) System and a method for monitoring

(76) Inventors: Avi BAR HAYIM, Maalot (IL); Diana EGOO, Karmiel (IL)

(21) Appl. No.: 12/826,721

(22) Filed: Jun. 30, 2010

Related U.S. Application Data
(63) Continuation-in-part of application No. PCT/IL2008/001698, filed on Dec. 30, 2008.
(60) Provisional application No. 61/018,450, filed on Jan. 1, 2008.

(51) Int. Cl. A61B 5/04

(52) U.S. Cl. 600/391

(57) ABSTRACT

A disposable monitor, including an adhesive sensing unit that includes a flexible conductive wire of length that does not exceed few centimeters; wherein when the adhesive sensing unit is attached to the skin of a person the flexible conductive wire changes an electrical parameter in response to movements of the skin; wherein the adhesive sensing unit wirelessly transmits electromagnetic signals that are indicative of values of the electrical parameter of the flexible conductive wire.

### RELATED APPLICATIONS

This application is a continuation in part of PCT patent application WO2009/083980 having an international filing date of Dec. 30, 2008, that in turn claims the priority of U.S. provisional patent Ser. No. 61/018,450 filing date 1 Jan. 2008, both being incorporated herein by reference.

### FIELD OF THE INVENTION

The invention relates to methods and systems for monitoring.

### BACKGROUND OF THE INVENTION

Monitoring physiological parameters of people, and especially of medical patients, is very important for both keeping the health of the person and usually also facilitates a substantial reduction of the work load of a hospital crew. Many prior art solutions for monitoring physiological parameters are either not sufficiently efficient or very expensive. It is noted that monitoring of different physiological parameters (e.g. breathing) is used not only in hospitals and in other medical situation, but also at domestic use, such as in monitoring physical parameters of a baby, a child, or an adult. There is a clear need to provide a low cost high reliability systems and method for monitoring.

### SUMMARY OF THE INVENTION

A disposable monitor, including an adhesive sensing unit that includes a flexible conductive wire of length that does not exceed few centimeters; wherein when the adhesive sensing unit is attached to the skin of a person the flexible conductive wire changes an electrical parameter in response to movements of the skin; wherein the adhesive sensing unit wirelessly transmits electromagnetic signals that are indicative of values of the electrical parameter of the flexible conductive wire.

A monitoring system including: a disposable monitor, including an adhesive sensing unit that includes a flexible conductive wire of length that does not exceed a centimeter; wherein when the adhesive sensing unit is attached to the skin of a person the flexible conductive wire changes an electrical parameter in response to movements of the skin; wherein the adhesive sensing unit wirelessly transmits electromagnetic signals that are indicative of values of the electrical parameter of the flexible conductive wire; and a processing unit configured to receive the electromagnetic signals and generate person health information in response to the received electromagnetic signals.

A monitoring system including: multiple disposable monitors, each disposable monitor includes an adhesive sensing unit that includes a short flexible conductive wire; wherein when the adhesive sensing unit is attached to the skin of a person the flexible conductive wire changes an electrical parameter in response to movements of the skin; wherein the adhesive sensing unit wirelessly transmits electromagnetic signals that are indicative of values of the electrical parameter of the flexible conductive wire; and a processing unit configured to receive the electromagnetic signals from the multiple disposable monitors and generate person health information in response to the received electromagnetic signals.

A disposable monitor, including an implantable sensing unit that includes a flexible conductive wire of length that does not exceed a centimeter; wherein when the implantable sensing unit is implanted in proximate to an organ of a person the flexible conductive wire changes an electrical parameter in response to movements of the organ; wherein the implantable sensing unit wirelessly transmits electromagnetic signals that are indicative of values of the electrical parameter of the flexible conductive wire.

A method for monitoring a person, the method includes: attaching an adhesive sensing unit to a skin of a person so that a flexible conductive wire of the adhesive sensing unit changes a value of an electrical parameter in response to movements of the skin; wherein the adhesive sensing unit includes a flexible conductive wire of length that does not exceed few centimeters; and wirelessly transmitting electromagnetic signals that are indicative of values of the electrical parameter of the flexible conductive wire.

A method for monitoring a person, including: attaching an adhesive sensing unit to a skin of a person, the adhesive sensing unit includes a flexible conductive wire of length that does not exceed a centimeter; wherein when the adhesive sensing unit is attached to the skin of a person the flexible conductive wire changes an electrical parameter in response to millimetric movements of the skin; wherein the adhesive sensing unit wirelessly transmits electromagnetic signals that are indicative of values of the electrical parameter of the flexible conductive wire; receiving by a processing unit the electromagnetic signals and generating, by the processing unit, person health information in response to the received electromagnetic signals.

A method for monitoring, the method includes: attaching to a skin of a person multiple disposable monitors, each disposable monitor includes an adhesive sensing unit that includes a short flexible conductive wire; wherein when the adhesive sensing unit is attached to the skin of a person the flexible conductive wire changes an electrical parameter in response to movements of the skin; wherein the adhesive sensing unit wirelessly transmits electromagnetic signals that are indicative of values of the electrical parameter of the flexible conductive wire; receiving, by a processing unit, the electromagnetic signals from the multiple disposable monitors; and generating by the processing unit person health information in response to the received electromagnetic signals.

A method for monitoring, the method including: implanting into a body of a person an implantable sensing unit that includes a flexible conductive wire of length that does not exceed a centimeter; wherein when the implantable sensing unit is implanted in proximate to an organ of a person the flexible conductive wire changes an electrical parameter in response to movements of the organ; and wirelessly transmitting electromagnetic signals that are indicative of values of the electrical parameter of the flexible conductive wire.

A method for disconnecting an umbilical cord, the method includes: attaching a monitoring unit to the umbilical cord, the monitoring unit is configured to detect pulsating of the umbilical cord; receiving from the monitoring unit an indication of umbilical cord pulsation stopping; and disconnecting the umbilical cord, after receiving the indication of the umbilical cord pulsation stopping.

### BRIEF DESCRIPTION OF THE DRAWINGS

The foregoing and other objects, features, and advantages of the present invention will become more apparent from the following detailed description when taken in conjunction with the accompanying drawings. In the drawings, similar reference characters denote similar elements throughout the different views, in which:

FIGS. 1A through 1D illustrate a disposable monitor, according to an embodiment of the invention;

FIG. 1E is a detailed view of a flexible conductive wire, according to an embodiment of the invention;

FIG. 2 illustrates a disposable monitor, according to an embodiment of the invention;

FIG. 3 illustrates a monitoring system, according to an embodiment of the invention

FIG. 4 illustrates a monitoring system, according to an embodiment of the invention;

FIGS. 5a and 5b illustrate a processing unit, according to an embodiment of the invention;

FIGS. 6, 7, 8, and 9 illustrate different methods for monitoring a person, according to different embodiments of the invention;

FIG. 10 illustrates a method for disconnecting an umbilical cord, according to an embodiment of the invention; and

FIGS. 11 and 12 illustrate a disposable monitor, according to an embodiment of the invention.

### DETAILED DESCRIPTION OF THE DRAWINGS

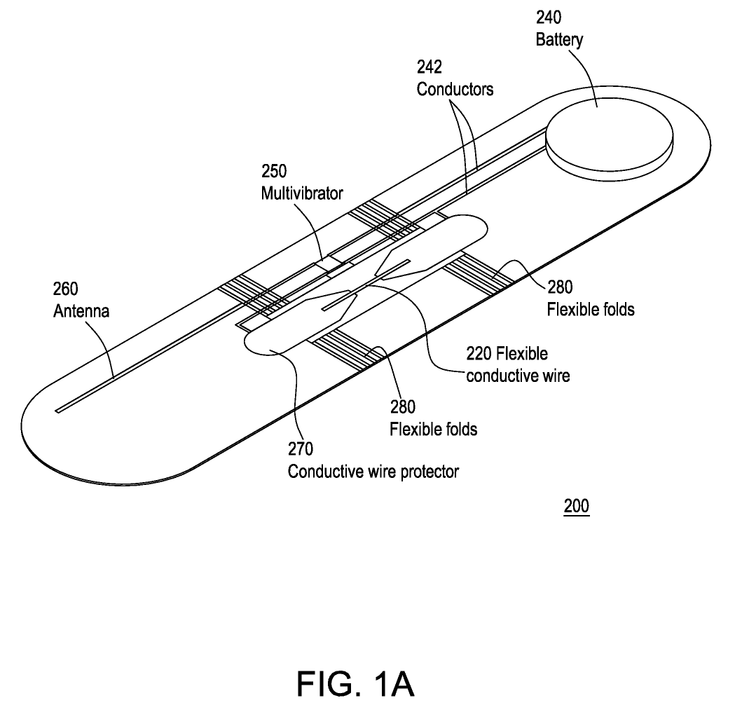

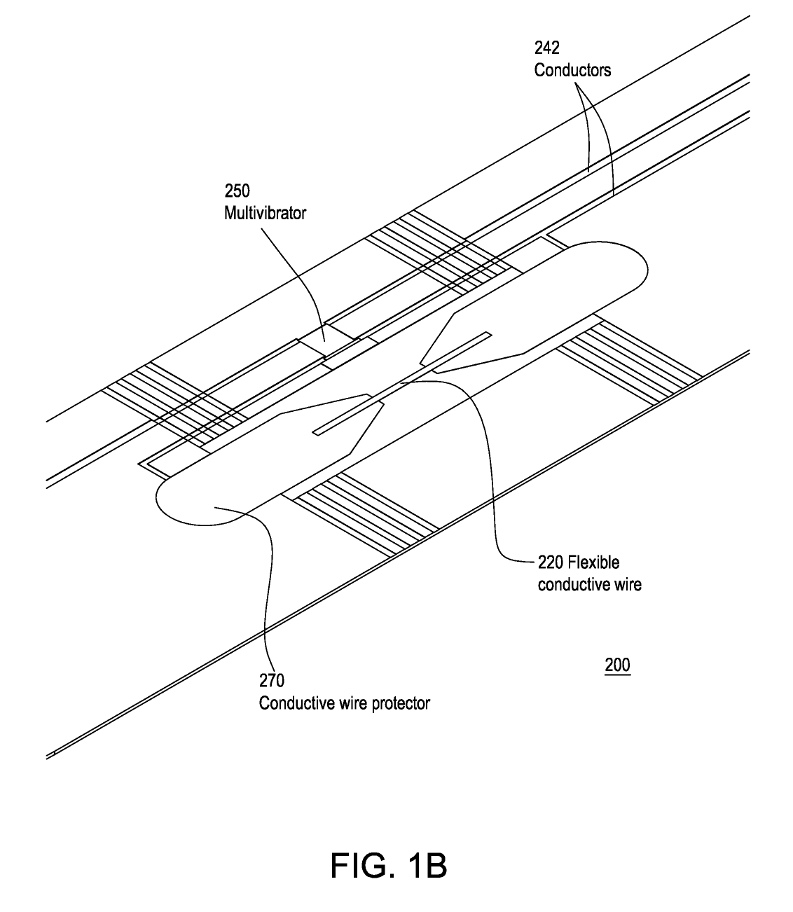

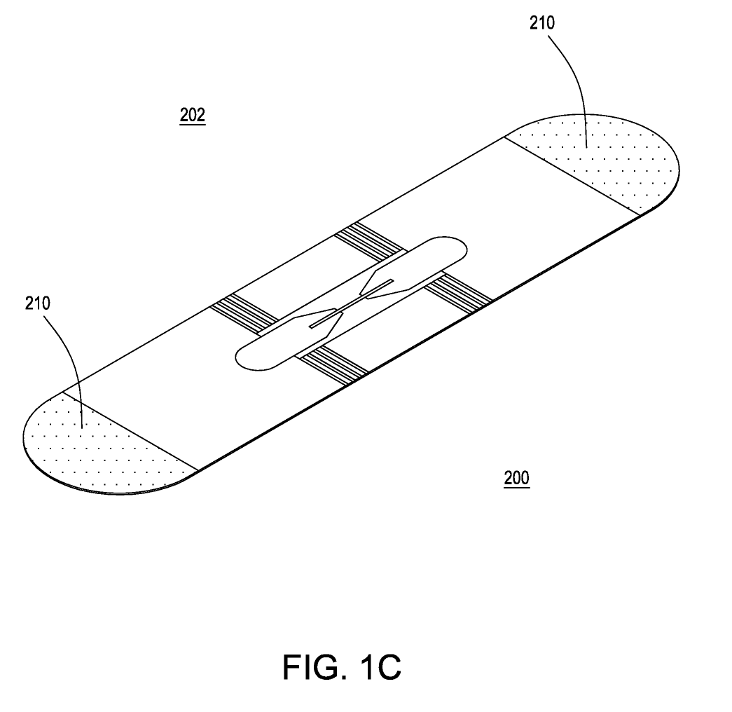

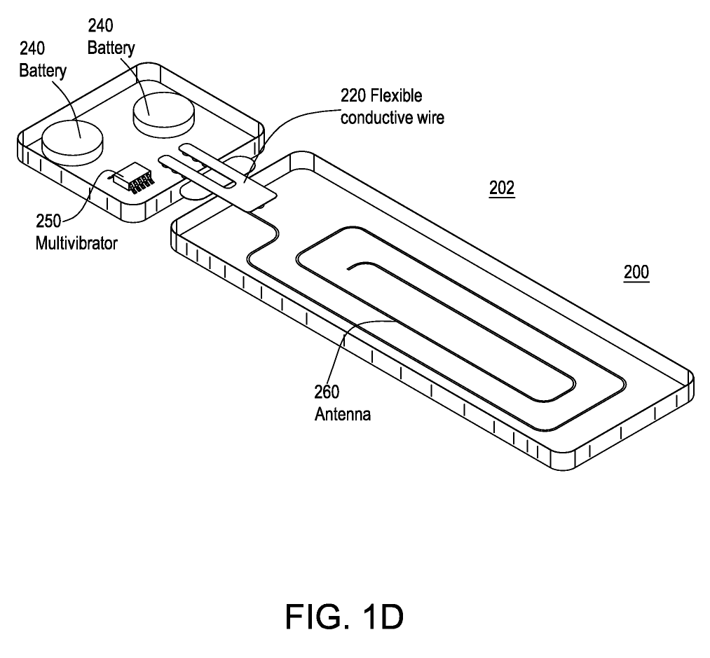

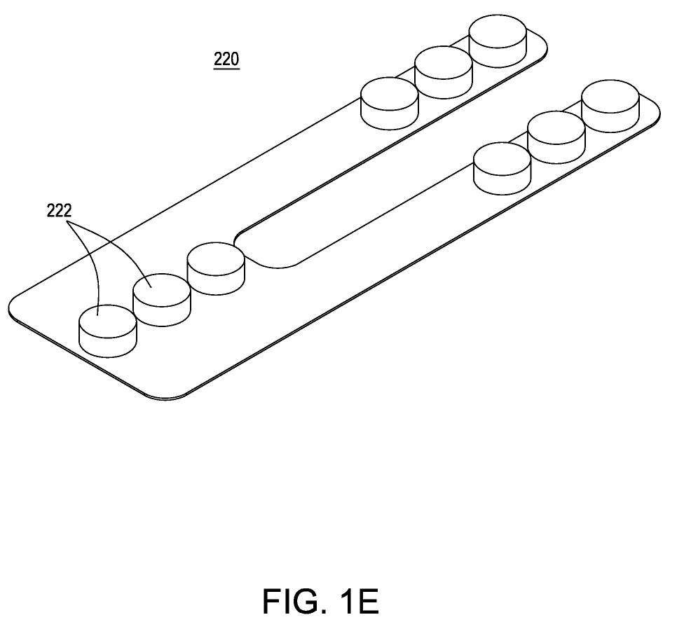

FIGS. 1A through 1D illustrate disposable monitor 202, according to an embodiment of the invention, wherein FIG. 1B is a close up view on a portion of adhesive sensing unit 200, FIG. 1C is a reverse view thereof, and FIG. 1D is a perspective view thereof, according to different embodiment of the invention. FIG. 1E is a detailed view of flexible conductive wire 220, according to an embodiment of the invention.

Disposable monitor 202 includes adhesive sensing unit 200 that includes flexible conductive wire 220 of length that does not exceed few centimeters; wherein when adhesive sensing unit 200 is attached to the skin of a person (not shown), flexible conductive wire 220 changes an electrical parameter in response to movements of the skin; wherein the adhesive sensing unit 200 wirelessly transmits electromagnetic signals that are indicative of values of the electrical parameter of the flexible conductive wire 220. Conveniently, the frequencies of the electromagnetic signals are indicative of the values of the electrical parameter but this is not necessarily so. It is noted that the content of the electromagnetic signals can indicate the value of the electrical parameter. For example they can convey one or more information fields indicative of the values of the electrical parameters.

Adhesive sensing unit 200 is conveniently adapted to be detachably attached to the skin of the person (usually by adhesive included in one or more adhesive surfaces 210 of adhesive sensing unit 200, e.g. like a standard adhesive bandage) for the monitoring of one or more physiological parameters of the person. Conveniently, flexible conductive wire 220 is connected (conveniently at its ends) to non-stretchable bands 230. conveniently, non-stretchable bands 210 are fixed to glue pads 212, which are conveniently about 10 cm apart from each other.

Conveniently, flexible conductive wire 220 is made from a material that an electrical parameter of which changes upon the applying of force on flexible conductive wire 220. e.g. according to an embodiment of the invention, the conductivity of flexible conductive wire 220 changes upon stretching and/or releasing flexible conductive wire 220. Conveniently, flexible conductive wire 220 is a conductive silicon wire.

Therefore, a voltage that is applied onto flexible conductive wire 220 (conveniently by battery 240 of adhesive sensing unit 200) results in a different current as the conductivity of flexible conducting wire 220 changes, as a result of force applied onto flexible conductive wire 220.

It is noted that different physiological parameters may be detected by movements of the skin, e.g. breathing, eyelids movement, penile erection, and so forth. Referring to breathing, for example, As the person breathes, the volume of one or more organs of the person (e.g. lung) increases and decreases, and therefore the skin of the person in the proximity of said organ stretches and is released. Since adhesive sensing unit 200 is detachably attached to the skin of the person by adhesive surfaces 210, the breathing of the person results in the stretching and releasing of flexible conductive wire 220, and hence to a change in the electrical parameter of flexible conductive wire 220, which is usually the resistance thereof.

Conveniently, when detachably attaching adhesive sensing unit 220 to the skin of the person, flexible conductive wire 220 is half stretched, allowing the skin to stretch further or to shrink back, thus, eliminating the need to wait for “The correct position for putting the adhesive sensing unit”.

It is noted that using pre-stretched flexible conductive wire 220 enables working in the midrange i.e. applying adhesive sensing unit 200 to the person in any position as long as it is on the monitored section of the body, or in proximity thereto (e.g. the breathing section of the body).

The changes in the resistance of flexible conductive wire 220 therefore results in a physiological related signal (e.g. breathing related signal), which may be converted into person health information (e.g. which includes information pertaining to a frequency of the breathing related signal). According to an embodiment of the invention, monitor 202 includes a multivibrator 250, (which is conveniently a free running multivibrator, like the 555 for example).

The voltage applied onto flexible conductive wire 220 is conveniently provided by battery 240 (e.g. by conductors 242). According to an embodiment of the invention, battery 240 is located in proximity to an adhesive surface, so as to increase a stability of adhesive sensing unit 200, and to decrease the chance of unintentional detaching of adhesive sensing unit 200 from the skin of the person.

According to an embodiment of the invention, battery 240 is a Lithium ion coin battery, which could provide the electricity needed for both the sensing and for the transmitting of the electromagnetic signals that are indicative of values of the electrical parameter of flexible conductive wire 220. It is noted that monitor 202 usually further includes an antenna 260, for wirelessly transmitting those electronic signals. According to an embodiment of the invention, antenna 260 is printed onto a surface of adhesive sensing unit 200.

According to an embodiment of the invention, adhesive sensing unit 200 includes at least one flexible portion (such as flexible folds 280) which is located in proximity to conductive wire 200, to facilitates the applying of the stretching of the skin of the person to flexible conductive wire 220.

According to an embodiment of the invention, adhesive sensing unit 200 includes at least one conductive wire protector 270, e.g. for protecting flexible conductive wire 220 from a mechanical impact.

According to an embodiment of the invention, flexible conductive wire 220 is made out of a thin conductive stretchable wire, such as conductive silicon. According to an embodiment of the invention, flexible conductive wire 220 is a 0.2 mm thick and 6 mm long piece of thin conductive stretchable wire.

According to an embodiment of the invention, flexible conductive wire 220 is of length that does not exceed a centimeter. According to an embodiment of the invention, flexible conductive wire 220 is of length that does not exceed six millimeters.

According to an embodiment of the invention, flexible conductive wire 220 is squaring the effect of elongation; as known to a person who is skilled in that art, the resistance R of conductive wire 240 equals r*L/A (where L is the length of flexible conductive wire 220, A is the cross sectional area of conductive wire 200, and r is the resistivity of the material of flexible conductive wire 220) and since silicon (or similar material) is not compressible, when the length of flexible conductive wire 220 increases, its area decreases respectively thus squaring the effect.

According to an embodiment of the invention, flexible conductive wire 220 is a very thin and short conductive wire, thus reducing the force needed to change its length, allowing making a very sensitive sensor with relatively low resistance, needed for low noise sensing.

It is noted that the application directly on the skin gives the best sensitivity to changes in it, the best isolation from the surroundings like closing, temp, humidity, etc.

Conveniently, the low profile of adhesive sensing unit 200 secures it from stacking objects and provide easiness in installation and comfort while using.

According to an embodiment of the invention, monitor 202 (And especially adhesive sensing unit 200) is water resistant and splash proofed.

By way of example, and not intending to limit the scope of the invention in any way, adhesive sensing unit 200 weighs 5 grams and costs around 0.1$ to produce.

According to an embodiment of the invention, the overall length of adhesive sensing unit 200 is 10 cm, and the width of adhesive sensing unit 200 is 2 cm. According to an embodiment of the invention, the distance between two adhesive surfaces 210 of adhesive sensing unit 200 us 7 cm.

According to an embodiment of the invention, adhesive sensing unit 200 includes additional biological sensors, for retrieving and providing to processing unit 300 additional information, pertaining, for example, to temp and heart pulse.

It is noted that monitor 202 (and adhesive sensing unit 200) may be used for different uses, only some of which are disclosed below. According to an embodiment of the invention, disposable monitor 202, when attached to a lid of the person, is configured to sense lid movements of the person. According to an embodiment of the invention, disposable monitor 202, when attached to a penis of a person, is configured to sense an erection.

According to an embodiment of the invention, disposable monitor 202, when attached to a umbilical cord, is configured to sense when the umbilical cord stops to pulsate. This may be used, for example, for disconnecting the umbilical cord only once it has stopped to pulsate.

According to an embodiment of the invention, disposable monitor 202, when attached to the skin of the person, is configured to sense vital signs of the person. Such vital signs may be, for example, breathing, pulse, movement of person, etc.

According to an embodiment of the invention, one or more of the above applications may be used for identifying a wakefulness state of the person (e.g. by identifying eyelids movement and pulse rate).

According to an embodiment of the invention, disposable monitor 202 includes a hermetic housing (not shown) that when opened exposes an adhesive surface of disposable monitor 202.

It is noted that, according to an embodiment of the invention, disposable monitor 202 is further adapted to wirelessly transmit additional information such as identification information, identifying the disposable monitor 202, the adhesive sensing unit 200, and so forth.

Referring to the scenario of breathing, for example, a 1%-10% (1 mm to 10 mm with 100 mm apart fixing points) expansion of the body skin while inhaling and exhaling 10 times, achieving 10% to 100% elongation of flexible conductive wire 220, that causes 12% to 400% increase in the resistance of flexible conductive wire 220 (for a silicon flexible conductive wire 220).

Referring to FIG. 1E, it is noted that according to an embodiment of the invention, flexible conductive wire 220 may have one or more contact elements 222, which may be used for physical contact and/or for electrical contact with other components of monitor 200.

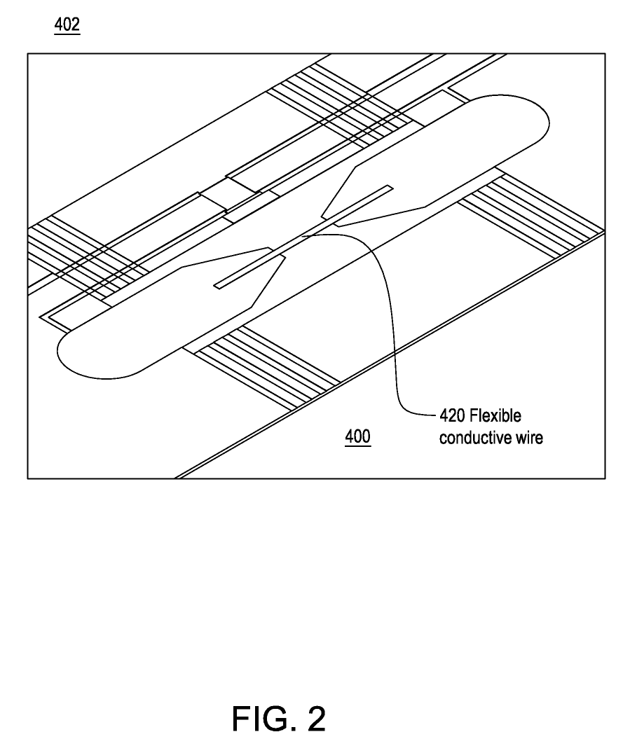

FIG. 2 illustrate disposable monitor 402, according to an embodiment of the invention. disposable monitor 402 includes an implantable sensing unit 400 that includes a flexible conductive wire 420 of length that does not exceed a centimeter; wherein when the implantable sensing unit 400 is implanted in proximate to an organ of a person the flexible conductive wire 420 changes an electrical parameter in response to movements of the organ; wherein the implantable sensing unit 400 wirelessly transmits electromagnetic signals that are indicative of values of the electrical parameter of the flexible conductive wire 420.

According to an embodiment of the invention, disposable monitor 402 is further adapted to wirelessly transmit identification information.

According to an embodiment of the invention, flexible conductive wire 420 is made of conductive silicone. It is noted that additional components, uses, and implementations of disposable monitor 402 may be similar to those of disposable monitor 202, mutatis mutandis.

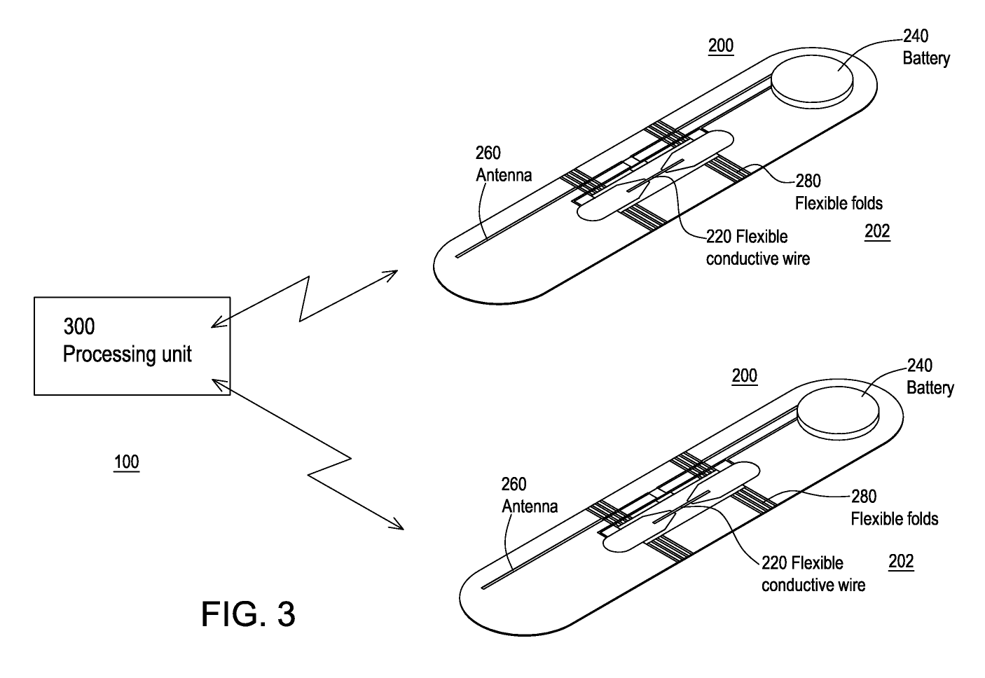

FIG. 3 illustrates monitoring system 100, according to an embodiment of the invention. Monitoring system 100 includes a disposable monitor (denoted 202, but may be either 202 or 402) that includes an adhesive sensing unit 200 (or alternatively an implementable sensing unit 400, also below) that includes a flexible conductive wire 220 (or 420) of length that does not exceed a centimeter; wherein when the adhesive sensing unit 200 is attached to the skin of a person the flexible conductive wire 220 changes an electrical parameter in response to movements of the skin; wherein the adhesive sensing unit 200 wirelessly transmits electromagnetic signals that are indicative of values of the electrical parameter of the flexible conductive wire 220; and monitor system 100 further includes processing unit 300 configured to receive the electromagnetic signals and to generate person health information in response to the received electromagnetic signals.

It is noted that, according to an embodiment of the invention, monitoring system 100 includes multiple disposable monitors 200; wherein the processing unit 300 is configured to receive electromagnetic signals from the multiple disposable monitors 200 and to generate health information in response to the received electromagnetic signals.

According to an embodiment of the invention, the processing unit 300 is configured to compare electromagnetic signals from different disposable monitors 200 and generate health information in response to the comparison.

According to an embodiment of the invention, processing unit 300 is configured to compare between electromagnetic signals of different disposable monitors 200 that are located at different distances from a heart of a person and are configured to movements of blood vessels of the person and provide an atherosclerosis indication.

According to an embodiment of the invention, processing unit 300 is configured to: (a) receive electromagnetic signals of a first disposable monitor 200 that monitors respiratory movement of a person; (b) receive electromagnetic signals of a second disposable monitor that monitors lid movements of the person; and (c) generate a fatigue indication in response to the received electromagnetic signals.

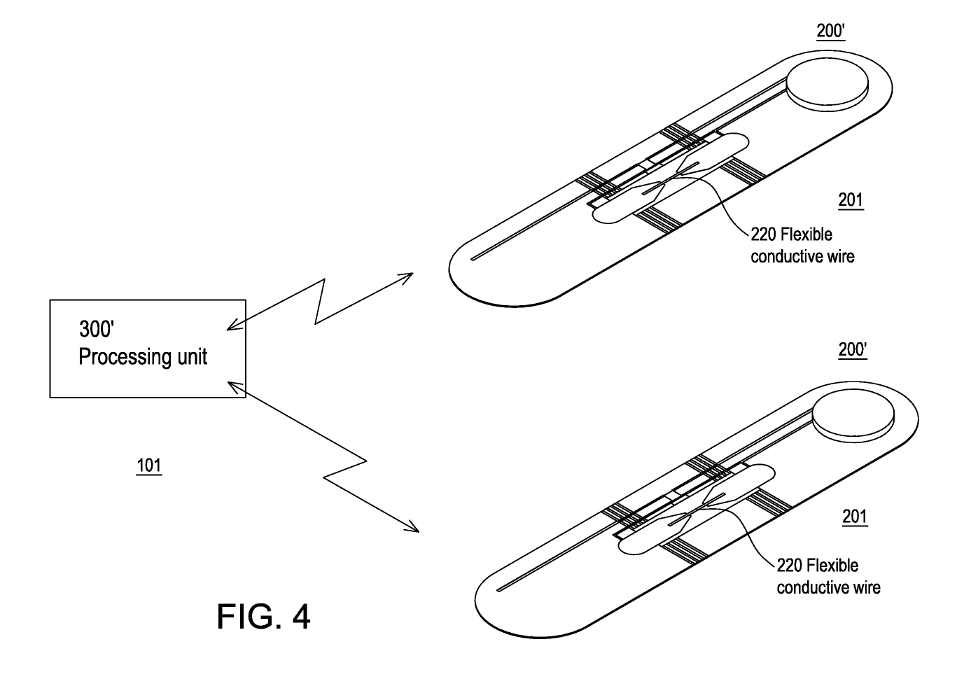

FIG. 4 illustrates monitoring system 101, according to an embodiment of the invention. monitoring system 101 includes multiple disposable monitors (denoted 201), each disposable monitor includes an adhesive sensing unit 200′ (or alternatively an implantable sensing unit) that includes a short flexible conductive wire 220′; wherein when the adhesive sensing unit 200′ is attached to the skin of a person the flexible conductive wire 220′ changes an electrical parameter in response to movements of the skin; wherein the adhesive sensing unit 200′ wirelessly transmits electromagnetic signals that are indicative of values of the electrical parameter of the flexible conductive wire 220′; and a processing unit 300′ configured to receive the electromagnetic signals from the multiple disposable monitors and generate person health information in response to the received electromagnetic signals.

According to an embodiment of the invention, processing unit 300′ is configured to compare electromagnetic signals from different disposable monitors 201 and to generate health information in response to the comparison.

According to an embodiment of the invention, processing unit 300′ is configured to compare between electromagnetic signals of different disposable monitors 201 that are located at different distances from a heart of a person and are configured to movements of blood vessels of the person and provide an atherosclerosis indication.

According to an embodiment of the invention, processing unit 300′ is configured to: (a) receive electromagnetic signals of a first disposable monitor 201 that monitors respiratory movement of a person; (B) receive electromagnetic signals of a second disposable monitor 201 that monitors lid movements of the person; and (c) Generate a fatigue indication in response to the received electromagnetic signals.

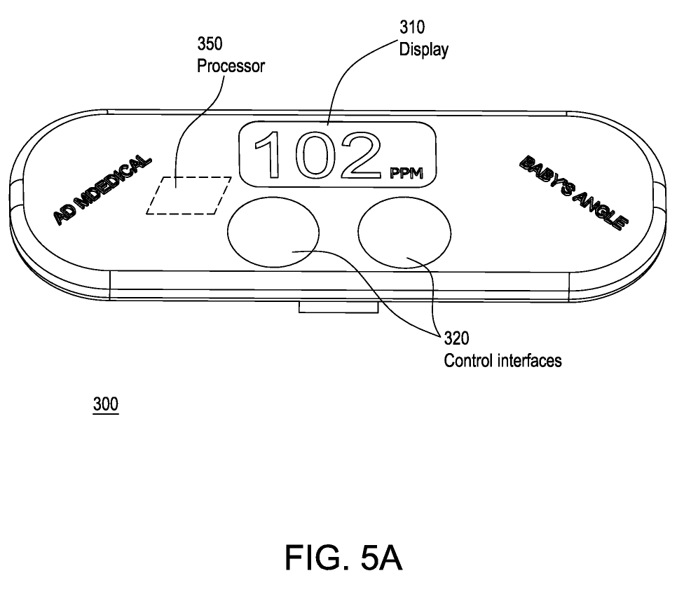

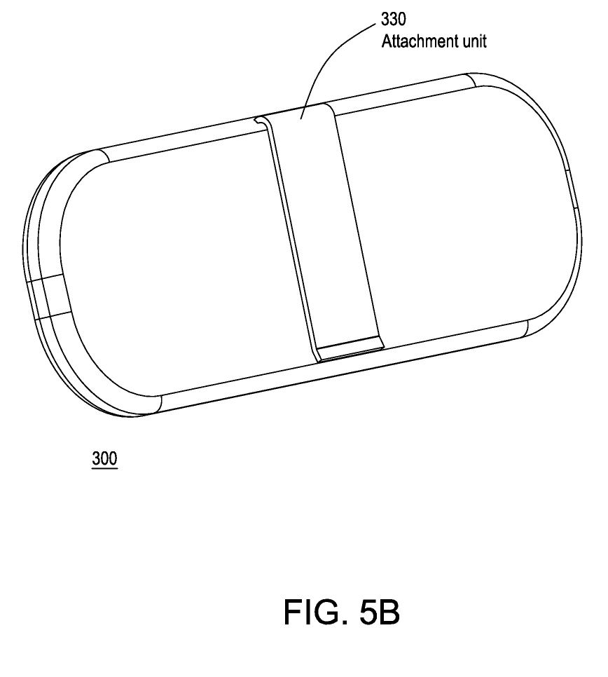

FIGS. 5a and 5b illustrate processing unit 300 (or 300′), according to an embodiment of the invention. Processing unit 300 includes a receiver, for receiving the breathing related information (e.g. a 50 KHz receiver), and processor 350 which is adapted to process the breathing related information to processed breathing related information. Conveniently, processor 350 is a digital signal processing (DSP) processor (e.g. an MSP430 family member), and conveniently a battery (such as a rechargeable battery, e.g. a lithium foil battery).

According to an embodiment of the invention, processing unit 300 further includes a standard PC communication interface, for transmitting the processed breathing information to external unit (e.g. USB or Bluetooth interface). It is noted that the external unit is, according to some different embodiments of the invention, a personal computer, a personal digital assistant (PDA), a cellular phone, or a control center unit at a hospital etc.

Conveniently, processor 350 is adapted to carry out programs for analyzing the processed breathing information, and for issuing alarms accordingly. (it is noted that according to an embodiment of the invention, processing unit further includes output interface, for the providing of alarms).

According to different embodiments of the invention, both local and remote alarms are possible, e.g. for single use at home or multiple users in hospitals or geriatric institutions.

It is noted that, conveniently, processing unit 300 is adapted to record the frequency pattern and to make breathing pattern related decisions using common knowledge breathing patterns.

According to an embodiment of the invention, processor 350 is adapted to compensate for auto bias using program, gets the bias line continually and allows freedom of use without special adjustments and special care.

According to an embodiment of the invention, system 100 facilitates a positive identification of breathing problem is 10 seconds.

It is noted that, conveniently, the data processing it computerized.

According to an embodiment of the invention, processing unit 300 includes a casing which includes at least one attachment unit 330 (such as a clamp) which is adapted to attach processing unit 300 to an external object (e.g. a bed of the user, a baby's stroller and so forth).

According to an embodiment of the invention, processing unit 300 includes display 310 for displaying breathing related data. According to an embodiment of the invention, processing unit 300 includes one or more control interfaces 320, for receiving orders from an operator of processing unit 300.

It is noted that in all of the systems above, wherever conductive silicon is implemented, according to an embodiment of the invention both conductive silicon and non-conductive silicon are used for manufacturing of the monitoring system. According to an embodiment of the invention, the manufacturing of a monitoring system such as the ones disclosed above includes preparing large plates of non-conductive silicon (that are relatively inexpensive), and cutting those place to receive multiple inexpensive adhesive surfaces. Following the cutting (or prior to the cutting), the conductive silicon may be inserted into the places (e.g. using Toluene—also known as methylbenzene—or similar chemical product). It is noted that conveniently, such process does not require metal electrical connections for connecting of the conductive silicon wire, thus reducing the price.

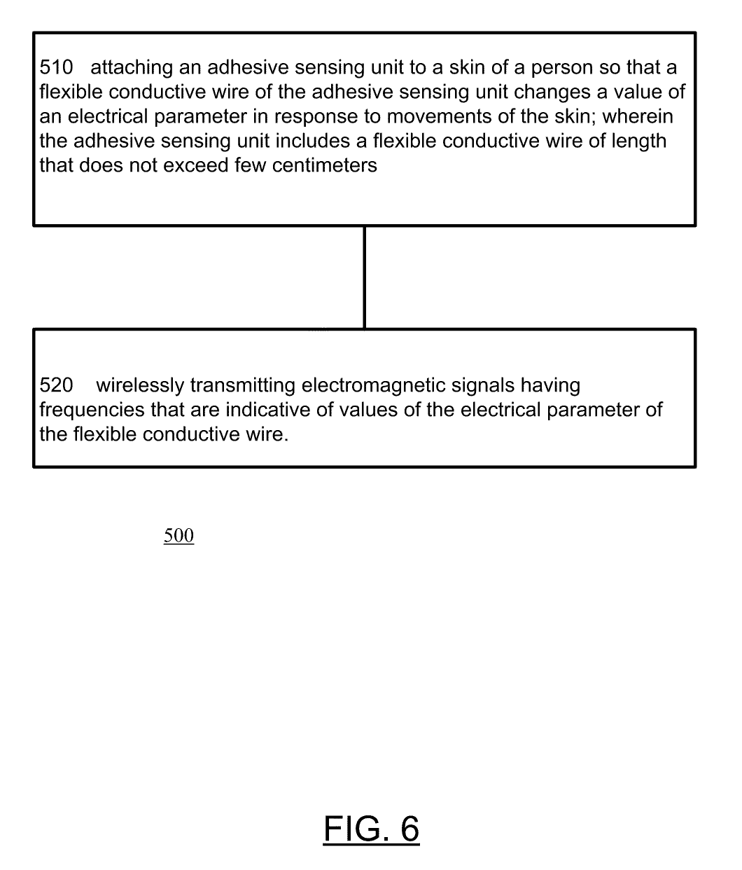

FIG. 6 illustrates method 500 for monitoring a person, according to an embodiment of the invention. method 500 starts with stage 510 of attaching an adhesive sensing unit to a skin of a person so that a flexible conductive wire of the adhesive sensing unit changes a value of an electrical parameter in response to movements of the skin; wherein the adhesive sensing unit includes a flexible conductive wire of length that does not exceed few centimeters. Stage 510 is followed by stage 520 of wirelessly transmitting electromagnetic signals that are indicative of values of the electrical parameter of the flexible conductive wire.

According to an embodiment of the invention, method 500 includes applying skin movement related forces on the flexible conductive wire by at least one flexible portion that is located in proximity to the flexible conductive wire.

According to an embodiment of the invention, method 500 includes sensing movements of the skin by a flexible conductive wire that is made of conductive silicone.

According to an embodiment of the invention, the attaching of method 500 includes attaching to a lid of the person at least a portion of the adhesive sensing unit that is configured to sense lid movements of the person.

According to an embodiment of the invention, the attaching of method 500 includes attaching to a skin of the person at least a portion of the adhesive sensing unit that is configured to sense vital signs of the person.

According to an embodiment of the invention, the attaching of method 500 includes attaching to a penis of the person at least a portion of the adhesive sensing unit that is configured to sense an erection.

According to an embodiment of the invention, the attaching of method 500 is preceded by opening a hermetic housing of the adhesive sensing unit that when opened exposes an adhesive surface of the disposable monitor.

According to an embodiment of the invention, the attaching of method 500 includes attaching to an umbilical cord at least a portion of the adhesive sensing unit that is configured to sense when the umbilical cord stops to pulsate.

According to an embodiment of the invention, method 500 further includes wirelessly transmitting identification information.

According to an embodiment of the invention, the attaching of method 500 includes attaching the adhesive sensing unit that includes the flexible conductive wire of length that does not exceed a centimeter.

According to an embodiment of the invention, the attaching of method 500 includes attaching the adhesive sensing unit that includes the flexible conductive wire of length that does not exceed six millimeters.

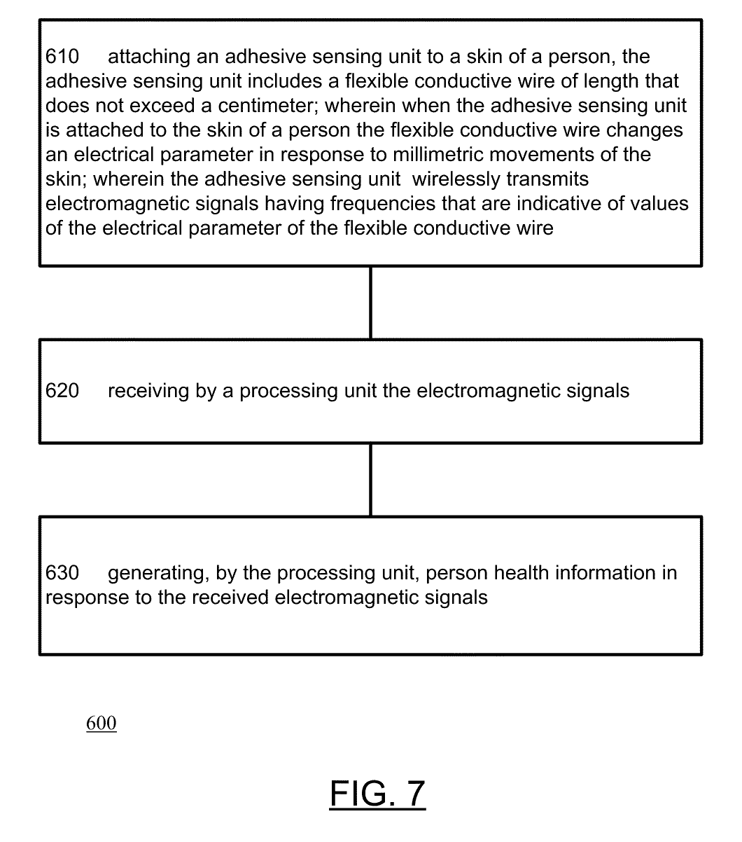

FIG. 7 illustrates method 600 for monitoring a person, according to an embodiment of the invention.

Method 600 starts with stage 610 of attaching an adhesive sensing unit to a skin of a person, the adhesive sensing unit includes a flexible conductive wire of length that does not exceed a centimeter; wherein when the adhesive sensing unit is attached to the skin of a person the flexible conductive wire changes an electrical parameter in response to millimetric movements of the skin; wherein the adhesive sensing unit wirelessly transmits electromagnetic signals that are indicative of values of the electrical parameter of the flexible conductive wire. Stage 610 is followed by stage 620 of receiving by a processing unit the electromagnetic signals, which is followed by stage 630 of generating, by the processing unit, person health information in response to the received electromagnetic signals.

According to an embodiment of the invention, the attaching of method 600 includes attaching multiple adhesive sensing units; wherein the receiving of method 600 includes receiving by the processing unit electromagnetic signals from the multiple disposable monitors and wherein the generating of method 600 includes generating health information in response to the received electromagnetic signals.

According to an embodiment of the invention, the generating of method 600 is preceded by comparing, by the processing unit, electromagnetic signals from different disposable monitors and wherein the generating of method 600 includes generating the health information in response to the comparison.

According to an embodiment of the invention, the comparing of method 600 includes comparing between electromagnetic signals of different disposable monitors that are located at different distances from a heart of a person and are configured to movements of blood vessels of the person and wherein the generating of method 600 includes providing an atherosclerosis indication.

According to an embodiment of the invention, the receiving of method 600 includes (a) receiving electromagnetic signals of a first disposable monitor that monitors respiratory movement of a person; and (b) receiving electromagnetic signals of a second disposable monitor that monitors lid movements of the person; and wherein the generating of method 600 includes generating a fatigue indication in response to the received electromagnetic signals.

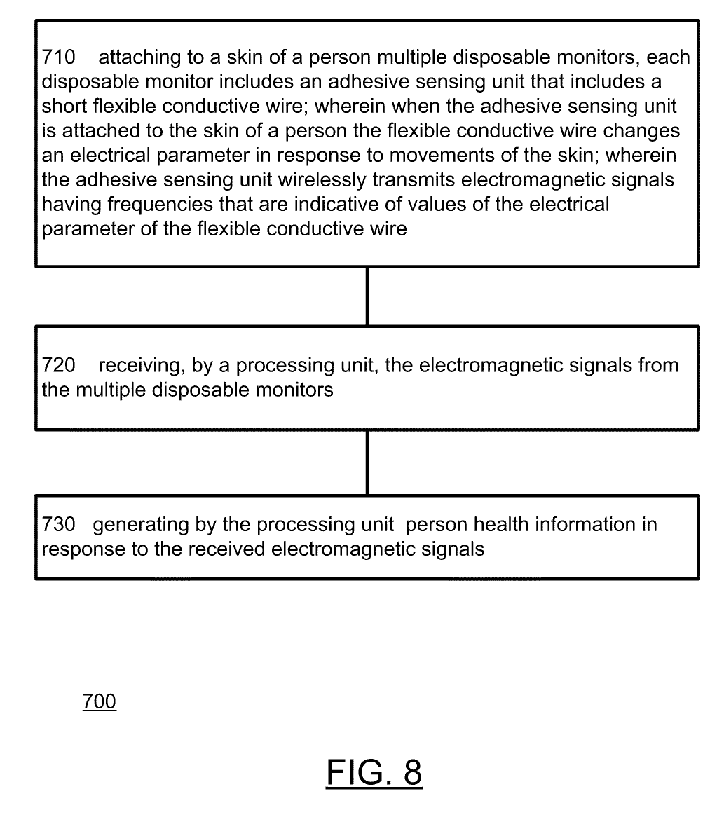

FIG. 8 illustrates method 700 for monitoring, according to an embodiment of the invention. method 700 starts with stage 710 of attaching to a skin of a person multiple disposable monitors, each disposable monitor includes an adhesive sensing unit that includes a short flexible conductive wire; wherein when the adhesive sensing unit is attached to the skin of a person the flexible conductive wire changes an electrical parameter in response to movements of the skin; wherein the adhesive sensing unit wirelessly transmits electromagnetic signals that are indicative of values of the electrical parameter of the flexible conductive wire. Stage 710 is followed by stage 720 of receiving, by a processing unit, the electromagnetic signals from the multiple disposable monitors; which is followed by stage 730 of generating by the processing unit person health information in response to the received electromagnetic signals.

According to an embodiment of the invention, the generating of method 700 is preceded by comparing electromagnetic signals from different disposable monitors and wherein the generating includes generating health information in response to the comparison.

According to an embodiment of the invention, the comparing of method 700 includes comparing between electromagnetic signals of different disposable monitors that are located at different distances from a heart of a person and are configured to movements of blood vessels of the person and wherein the generating of method 700 includes providing an atherosclerosis indication.

According to an embodiment of the invention, receiving of method 700 includes (a) receiving electromagnetic signals of a first disposable monitor that monitors respiratory movement of a person and (b) receiving electromagnetic signals of a second disposable monitor that monitors lid movements of the person; and wherein the generating of method 700 includes generating a fatigue indication in response to the received electromagnetic signals.

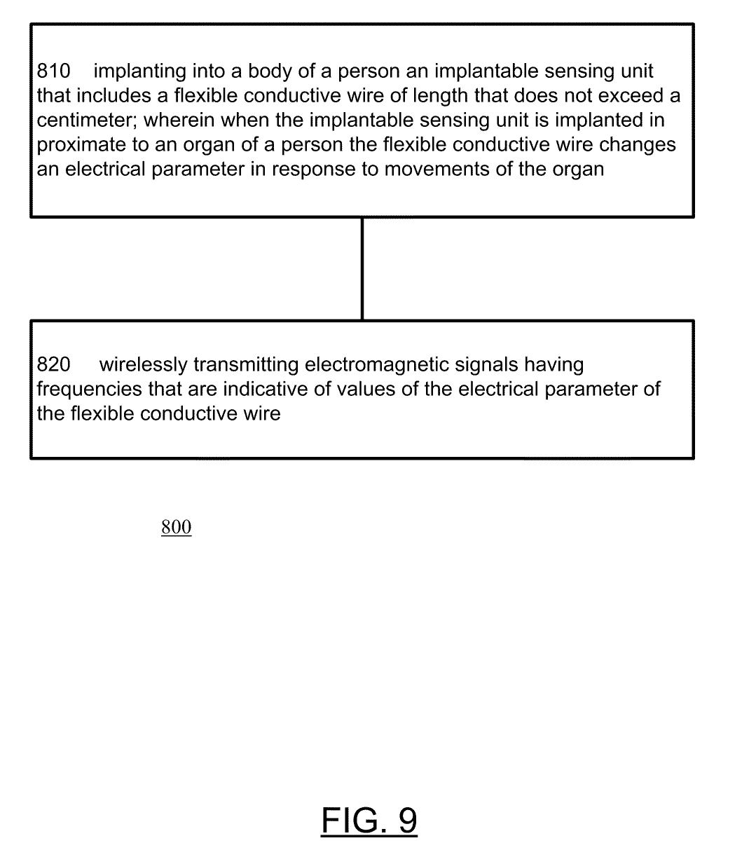

FIG. 9 illustrates method 800 for monitoring, according to an embodiment of the invention. method 800 includes stage 810 of implanting into a body of a person an implantable sensing unit that includes a flexible conductive wire of length that does not exceed a centimeter; wherein when the implantable sensing unit is implanted in proximate to an organ of a person the flexible conductive wire changes an electrical parameter in response to movements of the organ; that is followed by stage 820 of wirelessly transmitting electromagnetic signals that are indicative of values of the electrical parameter of the flexible conductive wire.

According to an embodiment of the invention, method 800 further includes wirelessly transmitting identification information.

According to an embodiment of the invention, the implanting includes implanting the implantable sensing unit, the flexible conductive wire of which is made of conductive silicone.

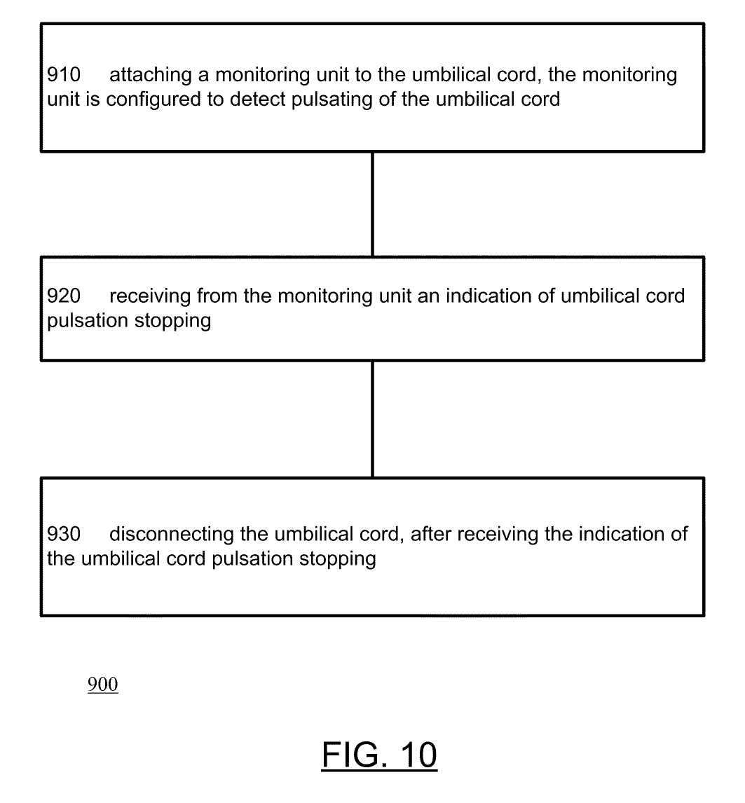

FIG. 10 illustrates method 900 for disconnecting an umbilical cord, according to an embodiment of the invention.

Method 900 starts with stage 910 of attaching a monitoring unit to the umbilical cord, the monitoring unit is configured to detect pulsating of the umbilical cord. Stage 910 is followed by stage 920 of receiving from the monitoring unit an indication of umbilical cord pulsation stopping. Stage 920 is followed by stage 930 of disconnecting the umbilical cord, after receiving the indication of the umbilical cord pulsation stopping.

According to an embodiment of the invention, stage 910 includes attaching a monitoring unit that includes an adhesive sensing unit that includes a flexible conductive wire of length that does not exceed few centimeters; wherein when the adhesive sensing unit is attached to the umbilical cord the flexible conductive wire changes an electrical parameter in response to movements of the umbilical cord; wherein the adhesive sensing unit wirelessly transmits electromagnetic signals that are indicative of values of the electrical parameter of the flexible conductive wire.

It is noted that method 900 may be implemented by systems 200 and 202 discussed above.

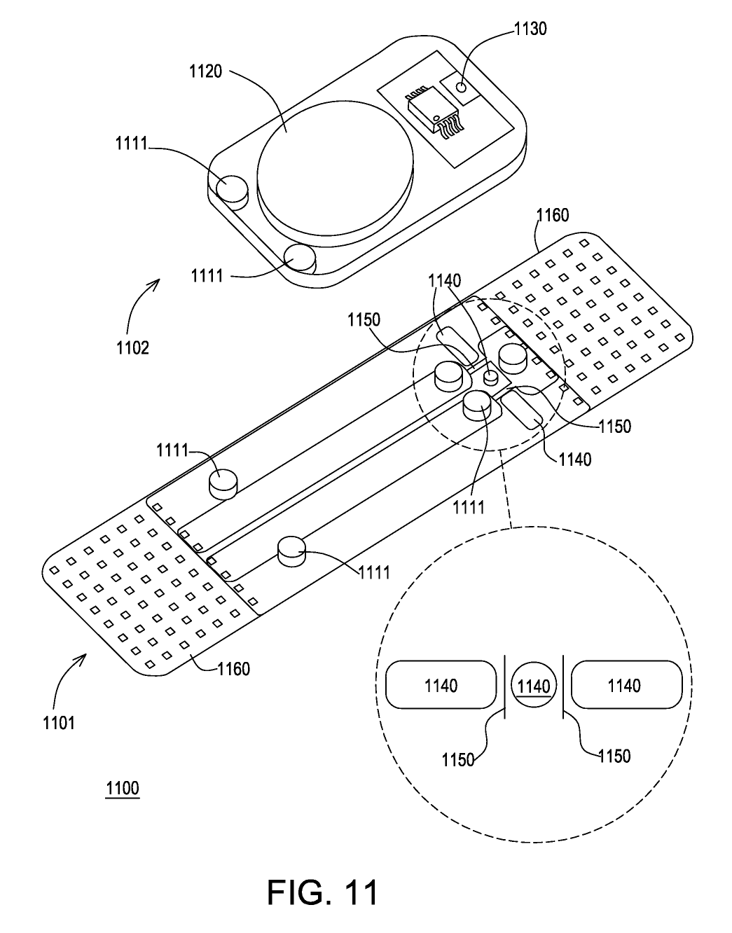

FIG. 11 illustrates a disposable monitor 1100, according to an embodiment of the invention.

The disposable monitor 1100 includes two portions—(i) a first portion 1101 that is attached to a person and may include components such as one or more flexible conductive wires 1150 and contacts 1111—at least some of which may electrically couple the flexible conductive wires 1150 to the second portion 1102, and (ii) a second portion 1102 that includes electrical components such as a battery 1120 and additional electrical components such as a multi-vibrator, and an antenna of a wireless transmitter. This separation allows re-using the second portion 1102 and may further reduce the cost of each test.

In the example set forth in FIG. 11, two flexible conductive wires 1150 are positioned between holes 1140 that may contribute to the sensitivity of the disposable monitor 110 by allowing the two flexible conductive wires 1150 to move in response to skin movement of a patient. The first portion is made of flexible material and has at its two opposing ends adhesive portions 1160.

The disposable monitor 1100 can apply (or at least participate in) any of the mentioned above methods.

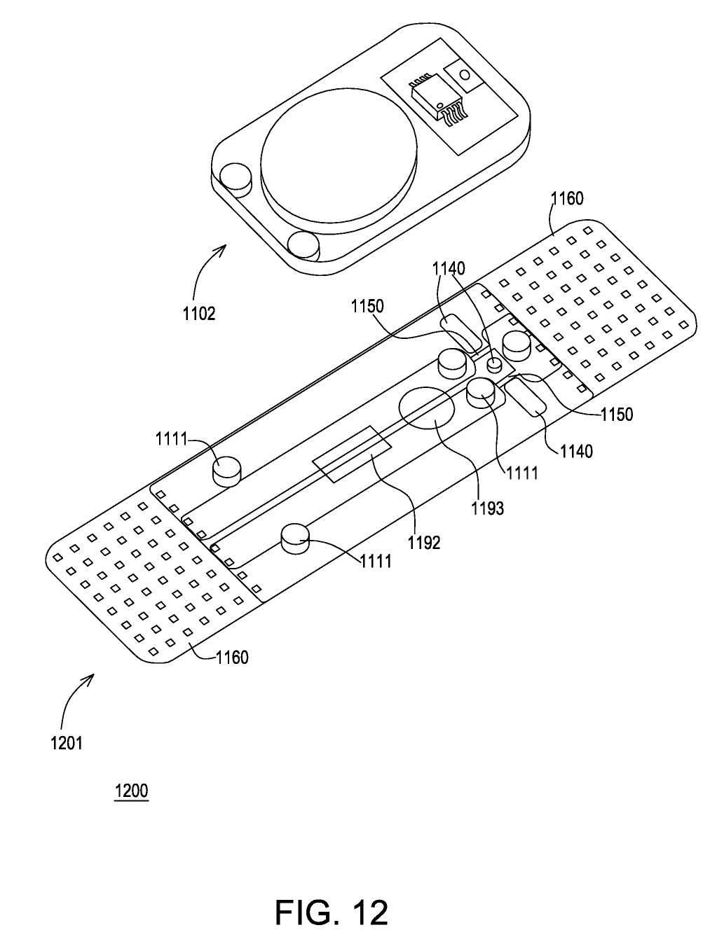

FIG. 12 illustrates a disposable monitor 1200 that includes a first portion 1021 and a second portion 1102. The first portion 1021 differs from the first portion of FIG. 11 by including additional components—a humidity sensor 1192 and a microphone 1193. It is noted that only one of these components can be a part of the disposable monitor.

Alternatively, at least one of these additional components (1192 and 1193) can be included in the second portion 1102 and not in the first portion 1202.

A microphone (1193 in FIG. 12) can be attached to or integrated within any of the mentioned above disposable monitors. The microphone 1193 can amplify sounds generated by the body of the patient such as heart beats, breath and the like.

A humidity sensor (1192 in FIG. 12) can be attached to or integrated within any of the mentioned above disposable monitors. The humidity detector and the flexible conductive wire, once attached to a patient can provide signals indicative of a tension or/and an excitement of a person and can be used as a low cost truth detector.

It is noted that the monitor can be used to evaluate the state of a patient, for example—determining if the patient is hydrated, is about to be hydrated (by detecting a weak heart beat), that is can be used to evaluate pain, and the like.

It is noted that any of the mentioned above disposable monitor can provide a long term of constant solution for monitoring, especially by using more durable materials.

It is noted that this separation can be applied to any of the previously disclosed systems or disposable monitors.

It is noted that disconnecting the umbilical cord only after cessation of pulsation may enable the newborn to receive larger amounts of essential materials such as blood, iron, oxygen, and so forth. It is noted that disconnecting the umbilical cord only after cessation of pulsation may enable an easier ejection of the placenta.

The present invention can be practiced by employing conventional tools, methodology and components. Accordingly, the details of such tools, component and methodology are not set forth herein in detail. In the previous descriptions, numerous specific details are set forth, in order to provide a thorough understanding of the present invention. However, it should be recognized that the present invention might be practiced without resorting to the details specifically set forth.

Only exemplary embodiments of the present invention and but a few examples of its versatility are shown and described in the present disclosure. It is to be understood that the present invention is capable of use in various other combinations and environments and is capable of changes or modifications within the scope of the inventive concept as expressed herein.

What is claimed is:

1. A monitoring system, comprising:
a disposable monitor, comprising an adhesive sensing unit that comprises a flexible conductive wire of length that does not exceed few centimeters;
wherein when the adhesive sensing unit is attached to the skin of a person the flexible conductive wire changes an electrical parameter in response to movements of the skin;
wherein the adhesive sensing unit is either arranged to wirelessly transmit electromagnetic signals or is arranged to be coupled to a wireless transmitter that is configured to wirelessly transmit electromagnetic signals, the wirelessly transmitted electromagnetic signals are indicative of values of the electrical parameter of the flexible conductive wire.

2. The monitoring system according to claim 1 that further comprises at least one flexible portion that is located in proximity to the flexible conductive wire to facilitate an applying of the stretching of the skin of the user to the flexible conductive wire.

3. The monitoring system according to claim 1 wherein the flexible conductive wire is made of conductive silicone.

4. The monitoring system according to claim 1 that when attached to a lid of the person it is configured to sense lid movements of the person.

5. The monitoring system according to claim 1 that when attached to a penis of a person it is configured to sense an erection.

6. The monitoring system according to claim 1 wherein the flexible conductive wire is of length that does not exceed six millimeters.

7. The monitoring system, according to claim 1, wherein:
the length of the flexible conductive wire does not exceed a centimeter; and
wherein the monitoring system further comprises a processing unit configured to receive the electromagnetic signals and generate person health information in response to the received electromagnetic signals.

8. The monitoring system according to claim 7 wherein the processing unit is configured to compare between electromagnetic signals of different disposable monitors that are located at different distances from a heart of a person and are configured to movements of blood vessels of the person and provide an atherosclerosis indication.

9. The monitoring system according to claim 7 wherein the processing unit is configured to:
receive electromagnetic signals of a first disposable monitor that monitors respiratory movement of a person;
receive electromagnetic signals of a second disposable monitor that monitors lid movements of the person; and
generate a fatigue indication in response to the received electromagnetic signals.

10. The monitoring system according to claim 1, comprising:
multiple disposable monitors, each disposable monitor comprises an adhesive sensing unit that comprises a short flexible conductive wire; wherein when the adhesive sensing unit is attached to the skin of a person the flexible conductive wire changes an electrical parameter in response to movements of the skin; wherein the adhesive sensing unit is either configured to wirelessly transmits electromagnetic signals or is arranged to be coupled to a wireless transmitter that is configured to wirelessly transmit the electromagnetic signals, wherein the electromagnetic signals are indicative of values of the electrical parameter of the flexible conductive wire; and
a processing unit configured to receive the electromagnetic signals from the multiple disposable monitors and generate person health information in response to the received electromagnetic signals.

11. The monitoring system according to claim 10 wherein the processing unit is configured to:
receive electromagnetic signals of a first disposable monitor that monitors respiratory movement of a person;
receive electromagnetic signals of a second disposable monitor that monitors lid movements of the person; and
generate a fatigue indication in response to the received electromagnetic signals.

12. A disposable monitor, comprising an implantable sensing unit that comprises a flexible conductive wire of length that does not exceed a centimeter; wherein when the implantable sensing unit is implanted in proximate to an organ of a person the flexible conductive wire changes an electrical parameter in response to movements of the organ; wherein the implantable sensing unit is configured to wirelessly transmit electromagnetic signals that are indicative of values of the electrical parameter of the flexible conductive wire.

13. A method for monitoring a person, the method comprises:
attaching an adhesive sensing unit to a skin of a person so that a flexible conductive wire of the adhesive sensing unit changes a value of an electrical parameter in response to movements of the skin; wherein the adhesive sensing unit comprises a flexible conductive wire of length that does not exceed few centimeters;
and
wirelessly transmitting electromagnetic signals that are indicative of values of the electrical parameter of the flexible conductive wire.

14. The method according to claim 13 comprising applying skin movement related forces on the flexible conductive wire by at least one flexible portion that is located in proximity to the flexible conductive wire.

15. The method according to claim 13 comprising sensing movements of the skin by a flexible conductive wire that is made of conductive silicone.

16. The method according to claim 13, wherein the attaching comprises attaching to a lid of the person at least a portion of the adhesive sensing unit that is configured to sense lid movements of the person.

17. The method according to claim 13, wherein the attaching comprises attaching to a penis of the person at least a portion of the adhesive sensing unit that is configured to sense an erection.

18. The method according to claim 13, wherein the attaching comprises attaching the adhesive sensing unit that comprises the flexible conductive wire of length that does not exceed six millimeters.

19. The method according to claim 13, wherein the length of the flexible conductive wire does not exceed a centimeter; wherein the method further comprises:
receiving by a processing unit the electromagnetic signals and
generating, by the processing unit, person health information in response to the received electromagnetic signals.

20. The method according to claim 19, wherein the attaching comprises attaching multiple adhesive sensing units; wherein the receiving comprises receiving by the processing unit electromagnetic signals from the multiple disposable monitors and wherein the generating comprises generating health information in response to the received electromagnetic signals.

21. The method according to claim 20, wherein comparing comprises comparing between electromagnetic signals of different disposable monitors that are located at different distances from a heart of a person and are configured to movements of blood vessels of the person and wherein the generating comprises providing an atherosclerosis indication.

22. The method according to claim 13, wherein the receiving comprises (a) receiving electromagnetic signals of a first disposable monitor that monitors respiratory movement of a person; and (b) receiving electromagnetic signals of a second disposable monitor that monitors lid movements of the person; and wherein the generating comprises generating a fatigue indication in response to the received electromagnetic signals.

23. The method according to claim 13, comprising: receiving, by a processing unit, the electromagnetic signals from the multiple disposable monitors; and generating by the processing unit person health information in response to the received electromagnetic signals.

24. A method for monitoring, the method comprising: implanting into a body of a person an implantable sensing unit that comprises a flexible conductive wire of length that does not exceed a centimeter; wherein when the implantable sensing unit is implanted in proximate to an organ of a person the flexible conductive wire changes an electrical parameter in response to movements of the organ; and wirelessly transmitting electromagnetic signals that are indicative of values of the electrical parameter of the flexible conductive wire.
